(19) United States
(12) Patent Application Publication
(10) Pub. No.: US 2025/0249061 A1
(43) Pub. Date: Aug. 7, 2025

(54) COMPOSITION AND SOFT CAPSULE FOR TREATING ATTENTION DEFICIT HYPERACTIVITY DISORDER IN CHILDREN, AND PREPARATION METHOD AND USE THEREOF

(71) Applicant: UNION THEORY LIMITED, Sha Tsui KL (CN)

(72) Inventors: Liang Chen, Sha Tsui KL (CN); Ye Zhou, Sha Tsui KL (CN); Xiaoyan Ma, Sha Tsui KL (CN)

(73) Assignee: UNION THEORY LIMITED, Sha Tsui KL (CN)

(21) Appl. No.: 18/668,889

(22) Filed: May 20, 2024

(30) Foreign Application Priority Data
Feb. 5, 2024 (CN) 202410166130.6

(51) Int. Cl. A61K 36/258; A61K 9/48; A61K 36/16; A61P 25/28

(52) U.S. Cl. CPC A61K 36/258; A61K 9/485; A61K 9/4858; A61K 9/4875; A61K 36/16; A61P 25/28

(57) ABSTRACT

The present disclosure belongs to the technical field of drug preparation, in particular to a composition and a soft capsule for treating attention deficit hyperactivity disorder (ADHD) in children, and their preparation method and use. In the present disclosure, phosphatidylserine is used as the core active ingredients. On this basis, ginkgo leaves extract and red ginseng extract are selected as supplementary active ingredients.

### CROSS-REFERENCE TO RELATED APPLICATION

This patent application claims the benefit and priority of Chinese Patent Application No. 202410166130.6 filed with the China National Intellectual Property Administration on Feb. 5, 2024, the disclosure of which is incorporated by reference herein in its entirety as part of the present application.

### TECHNICAL FIELD

The present disclosure belongs to the technical field of drug preparation, in particular to a composition and a soft capsule for treating attention deficit hyperactivity disorder in children, and a preparation method and use thereof.

### BACKGROUND

Attention-Deficit/Hyperactivity Disorder (ADHD) in children has attracted great attention from various sub-disciplines of pediatrics, such as developmental behavior, neurology and psychiatry. In 2006, diagnosis and treatment for attention-deficit/hyperactivity disorder in children were published in Chinese Journal of Pediatrics, which standardized the clinical diagnosis and treatment procedures of ADHD, and greatly promoted the rigor and scientificity of the diagnosis and treatment of this disease. As far as the treatment of ADHD, experts at home and abroad have reached the following consensus: (1) ADHD is a chronic disease that requires long-term treatment; (2) the comprehensive treatment of ADHD includes drug therapy and behavioral therapy according to evidence-based medicine; (3) stimulants are the drug of first choice; and (4) the treatment of ADHD should involve doctors, children, parents or caregivers, and teachers.

The treatment of ADHD is mainly a combination of medication and behavior intervention. Stimulants are one of the main therapeutic drugs for ADHD, including amphetamine, methylphenidate hydrochloride and pemoline. Stimulant therapy can quickly improve the behavior, attention and schoolwork of children and adolescents with ADHD. Stimulant therapy can effectively respond in about 70%- 80% of children with ADHD in a short period of time, improve the core symptoms of ADHD, such as attention problems, hyperactivity and impulsivity, and can also improve children's defiant and aggressive behavior to promote social interaction. However, the side effects of stimulants in the treatment of ADHD have always been concerned. The most common side effects are anorexia, sleep disorder, headache, motor tics, abdominal pain, irritability, nausea, and fatigue. Based on the harm of these side effects to children, many parents choose the combination of psychological and behavioral intervention to reduce drug intake. However, the improvement of the core symptoms of ADHD by psychobehavioral intervention is not ideal. The reasons may be as follows: (1) behavioral therapy takes time and needs the cooperation of family and school, and (2) the response of children with ADHD to behavioral therapy is slow in the disease state. Therefore, there is an urgent need for ADHD therapeutic drugs that can reduce or replace the intake of stimulants, so as to improve the therapeutic effect of ADHD and avoid the side effects of stimulants in ADHD treatment.

### SUMMARY

The purpose of the present disclosure is to provide a composition and a soft capsule for treating attention deficit hyperactivity disorder in children, and a preparation method and use thereof. The composition has a good therapeutic effect on attention deficit hyperactivity disorder in children, has a good improvement effect on various mal-adaptations of children with attention deficit hyperactivity disorder, and can reduce the use of stimulants and the incidence of various side effects.

The present disclosure provides a composition, including the following components in parts by mass: 1-50 parts of phosphatidylserine, 1-200 parts of ginkgo leaves extract, and 1-1,000 parts of red ginseng extract.

In some embodiments, a purity of phosphatidylserine is at least 20%.

In some embodiments, the ginkgo leaves extract includes ginkgo flavonoid and ginkgolide, and a mass concentration of ginkgo flavonoid is 24%, and a mass concentration of ginkgolide is 6%.

In some embodiments, the red ginseng extract includes Korean red ginseng extract.

The present disclosure further provides a soft capsule, including the composition described in the above technical scheme and soft capsule excipient, wherein a mass ratio of phosphatidylserine to soft capsule excipient is (1-50): (0-650), and the mass of soft capsule excipient is not 0;

The soft capsule excipient includes flaxseed oil, beeswax, gelatin, glycerol, water, titanium dioxide and pigment.

In some embodiments, in parts by mass, the soft capsule excipient includes 0-250 parts of flaxseed oil, 0-20 parts of beeswax, 0-210 parts of gelatin, 0-100 parts of glycerol, 0-38 parts of water, 0-3.50 parts of titanium dioxide and 0.42 parts of pigment. The pigment includes cocoa shell pigment; the mass of flaxseed oil, beeswax, gelatin, glycerol, water, titanium dioxide and pigment is not 0.

The present disclosure further provides a preparation method of the soft capsule according to the technical scheme, comprising the following steps:
mixing phosphatidylserine, ginkgo leaves extract powder and red ginseng extract powder to obtain a mixture;
mixing the mixture, flaxseed oil and beeswax to obtain an oily mixture;
mixing gelatin, glycerol, water, titanium dioxide and pigment to obtain a glue solution; and
thermally encapsulating the oily mixture with the glue solution and cooling to obtain the soft capsule.

The present disclosure further provides use of the composition described in the above technical scheme, the soft capsule described in the above technical scheme or the soft capsule prepared by the preparation method in the above technical scheme in the preparation of drugs for treating or adjuvant treating attention deficit hyperactivity disorder in children.

In some embodiments, the drugs include drugs with one or more functions of maintaining nervous system, anti-inflammatory, improving body energy metabolism, improving mood anxiety, repairing neurotransmitter conduction, improving learning and memory ability, and alleviating the mal-adaptations of attention deficit hyperactivity disorder in children.

The alleviation of the mal-adaptation symptoms of attention deficit hyperactivity disorder in children includes one or more of improving attention, adjusting emotion, improving hyperactivity symptoms, improving impulsive symptoms and reducing aggression.

The present disclosure further provides a combined drug for treating attention deficit hyperactivity disorder in children, the combined drug includes a stimulant drug and the composition described in the above technical scheme, the soft capsule described in the above technical scheme or the soft capsule prepared by the preparation method described in the above technical scheme.

### Beneficial Effects:

The present disclosure provides a composition, including the following components in parts by mass: 1-50 parts of phosphatidylserine, 1-200 parts of ginkgo leaves extract, and 1-1000 parts of red ginseng extract. In the present disclosure, phosphatidylserine is used as the core active ingredients, and is able to achieve the functions of maintaining the nervous system, repairing basic functions and reducing inflammation. On this basis, ginkgo leaves extract and red ginseng extract are selected as supplementary active ingredients to effectively alleviate the symptoms of attention deficit hyperactivity disorder (ADHD) in children. Various components in the soft capsule can synergistically supplement essential trace elements and nutrients for human body, and improve energy metabolism and anxiety. Test results show that the composition of the present disclosure can significantly improve the degree of spontaneous activity, social preference and irritability of rats, and improve the learning and memory ability of rats.

Meanwhile, the raw materials selected in the composition formula of the present disclosure are all natural active ingredients, which do not produce side effects on human bodies or animals, and the selected raw materials are green and safe, and will not cause pollution to the environment.

### DETAILED DESCRIPTION OF THE EMBODIMENTS

The present disclosure provides a composition, including the following components in parts by mass: 1-50 parts of phosphatidylserine, 1-200 parts of ginkgo leaves extract, and 1-1000 parts of red ginseng extract.

The composition includes 1-50 parts by mass of phosphatidylserine, preferably 25-50 parts by mass. The purity of phosphatidylserine is preferably at least 20%, more preferably 20%.

The phosphatidylserine of the present disclosure is the core active ingredients of the soft capsule, and is able to achieve the functions of maintaining the nervous system, repairing basic functions and reducing inflammation.

The composition of the present disclosure includes 1-200 parts of ginkgo leaves extract, preferably 50-100 parts, more preferably 62.5-80 parts, based on the mass parts of phosphatidylserine. The ginkgo leaves extract preferably includes ginkgo flavonoid and ginkgolide, and the mass concentration of ginkgo flavonoid in the ginkgo leaves extract is preferably 24%, and the mass concentration of ginkgolide in the ginkgo leaves extract is preferably 6%. There is no particular limitation on the source of the ginkgo leaves extract, a conventional extracting method or purchase will do.

Based on the mass parts of phosphatidylserine, the composition of the present disclosure includes 1-1,000 parts of red ginseng extract, preferably 200-600 parts, more preferably 250-400 parts. The red ginseng extract preferably includes Korean red ginseng extract; The Korean Red Ginseng extract is preferably Korean red ginseng extract.

The ginkgo leaves extract and the red ginseng extract of the present disclosure are used as supplementary raw material components of the soft capsule, which can effectively alleviate the symptoms of ADHD symptoms.

The present disclosure also provides a soft capsule including the composition described in the above technical scheme and soft capsule excipient, wherein the mass ratio of phosphatidylserine to soft capsule excipient is (1-50): (0-650), and the mass of soft capsule excipient is not 0;

The soft capsule excipients include flaxseed oil, beeswax, gelatin, glycerol, water, titanium dioxide and pigment.

The soft capsule of the present disclosure includes 0-650 parts of soft capsule excipient, preferably 624.92 parts, based on the mass parts of phosphatidylserine.

Based on the mass parts of phosphatidylserine, the soft capsule excipient of the present disclosure preferably includes 0-250 parts of flaxseed oil (linseed oil), more preferably 250 parts; preferably includes 0-20 parts of beeswax, more preferably 20 parts; preferably includes 0-210 parts of gelatin, more preferably 210 parts; preferably includes 0-100 parts of glycerol, more preferably 100 parts; preferably includes 0-38 parts of water, more preferably 38 parts; preferably includes 0-3.50 parts of titanium dioxide, more preferably 3.50 parts; preferably includes 0-0.42 part of pigment, more preferably 0.42 part. The pigment preferably includes cocoa shell pigment. The mass of flaxseed oil, beeswax, gelatin, glycerol, water, titanium dioxide and pigment is not 0.

The present disclosure also provides a preparation method of the soft capsule of the above technical scheme, comprising the following steps:
mixing phosphatidylserine, ginkgo leaves extract powder and red ginseng extract powder to obtain a mixture;
mixing the mixture, flaxseed oil and beeswax to obtain an oily mixture;
mixing gelatin, glycerol, water, titanium dioxide and pigment to obtain a glue solution;
thermally encapsulating the oily mixture with the glue solution and cooling to obtain the soft capsule.

In the present disclosure, the ginkgo leaves extract and the red ginseng extract are preferably ground into powder and then sieved to obtain the ginkgo leaves extract powder and the red ginseng extract powder respectively; and the pore size of the sieve hole for sieving is preferably 60 meshes.

After the ginkgo leaves extract powder and the red ginseng extract powder are obtained, phosphatidylserine, ginkgo leaves extract powder and red ginseng extract powder are mixed. There is no particular limitation on the mixing step, a conventional mixing step in the art will do.

After the mixing, the method of the present disclosure preferably further includes sieving the mixed materials to obtain a mixture. The pore size of the sieve hole for sieving is preferably 60 meshes.

After obtaining the mixture, the mixture, flaxseed oil and beeswax are mixed to obtain an oily mixture. There is no particular limitation on the mixing step, as long as it is uniform.

In the present disclosure, gelatin, glycerol, water, titanium dioxide and pigment are mixed to obtain glue solution. There is no particular limitation on the process of mixing the gelatin, glycerol, water, titanium dioxide and pigment, a conventional mixing step in the art will do.

After the glue solution and oily mixture are obtained, the oily mixture is thermal encapsulated with the glue solution and then cooled to obtain the soft capsule. In the present disclosure, a pill dropping machine is preferably used to prepare the soft capsule, and the method specifically preferably includes the steps of: spraying the glue solution and the oily mixture at different speeds through the nozzle of the pill dropping machine, encapsulating the oily mixture with the glue solution and then dripping the resulting encapsulated glue solution into the coolant to prepare the soft capsule. There is no particular limitation on the specific process and parameters, the parameters of conventional soft capsule preparation in the art will do. The coolant preferably includes a liquid which is immiscible with the glue, and more preferably includes but not limited to liquid paraffin. The glue solution forms a sphere in the coolant due to surface tension and gradually solidifies into soft capsules.

In the present disclosure, phosphatidylserine is used as the core active ingredients, and is able to achieve the functions of maintaining the nervous system, repairing basic functions and reducing inflammation. On this basis, ginkgo leaves extract and red ginseng extract are selected as supplementary active ingredients to effectively alleviate the symptoms of attention deficit hyperactivity disorder (ADHD) in children. Various components in the soft capsule can synergistically supplement essential trace elements and nutrients for human body, and improve energy metabolism and anxiety. Meanwhile, the raw materials selected in the composition formula of the present disclosure are all natural active ingredients, which do not produce side effects on human bodies or animals, and the selected raw materials are green and safe, and will not cause pollution to the environment.

Based on the above technical advantages, the present disclosure also provides use of the composition described in the above technical scheme, the soft capsule described in the above technical scheme or the soft capsule prepared by the preparation method in the above technical scheme in the preparation of drugs for treating or adjuvant treating attention deficit hyperactivity disorder in children. The drugs include drugs with one or more functions of maintaining nervous system, anti-inflammatory, improving body energy metabolism, improving mood anxiety, repairing neurotransmitter conduction, improving learning and memory ability, and alleviating the mal-adaptations of attention deficit hyperactivity disorder in children, and more preferably is drugs with multiple functions of maintaining nervous system, anti-inflammatory, improving body energy metabolism, improving mood anxiety, repairing neurotransmitter conduction, improving learning and memory ability, and alleviating the mal-adaptations of attention deficit hyperactivity disorder in children. The alleviation of the various mal-adaptation symptoms of attention deficit hyperactivity disorder in children includes one or more of improving attention, adjusting emotion, improving hyperactivity symptoms, improving impulsive symptoms and reducing aggression, more preferably includes improving attention, adjusting emotion, improving hyperactivity symptoms, improving impulsive symptoms and reducing aggression.

The present disclosure also provides a combined drug for treating attention deficit hyperactivity disorder in children, the combined drug includes a stimulant drug and the soft capsule described in the above technical scheme or the soft capsule prepared by the preparation method described in the above technical scheme. The soft capsule of the present disclosure can be used alone or in combination with stimulant drugs for treating attention deficit hyperactivity disorder in children, so as to achieve the effect of adjuvant treatment or treatment of attention deficit hyperactivity disorder in children, and at the same time, reduce the dosage of stimulant drugs and reduce their side effects.

In order to further illustrate the present disclosure, the technical schemes provided by the present disclosure will be described in detail below with reference to examples, but they should not be understood as a limitation to the protection scope of the present disclosure.

### EXAMPLE 1

A composition for treating attention deficit hyperactivity disorder in children, including the following components in parts by mass:
50 parts of phosphatidylserine (20%), 62.5 parts of ginkgo leaves extract (ginkgo flavonoid 24% and ginkgolide 6%) and 250 parts of Korean red ginseng extract.

The preparation steps of ginkgo leaves extract were as follows: 0.15 g of ginkgo biloba leaves were taken and weighed accurately, 10 mL of water was added, the weighed ginkgo biloba leaves and water were placed in a water bath and heated to dissolve and disperse, 2 drops of 2% hydrochloric acid solution were added to adjust the pH value of the solution to weak acid, the resulting weak acid solution was shaken and extracted with ethyl acetate for four times at room temperature (the amount of ethyl acetate extracted by four times was 15 mL, 10 mL, 10 mL, 10 mL respectively), the extracts were combined and washed with 20 mL of 5% sodium acetate solution. The sodium acetate layer was retained, followed by washing with 10 mL of ethyl acetate to obtain the ginkgo leaves extract, in which the content of ginkgo flavonoid was 24% and the content of ginkgolide was 6%.

The preparation steps of Korean red ginseng extract were as follows: Korean red ginseng was pulverized into coarse powder, the coarse powder was extracted with ethanol reflux solution with a volume concentration of 60%- 70% at 60°° C., the obtained extract was concentrated under reduced pressure to a liquid extract of about 15 Baume (thermal measurement), and the liquid extract was spray-dried to obtain dry powder, the Korean red ginseng extract was obtained.

### EXAMPLE 2

A composition for treating attention deficit hyperactivity disorder in children, including the following components in parts by mass:
10 parts of phosphatidylserine (20%), 200 parts of ginkgo leaves extract (ginkgo flavonoid 24% and ginkgolide 6%) and 1 parts of Korean red ginseng extract.

The preparation methods of ginkgo leaves extract and Korean red ginseng extract were the same as in Example 1.

### EXAMPLE 3

A composition for treating attention deficit hyperactivity disorder in children, including the following components in parts by mass:
1 parts of phosphatidylserine (20%), 100 parts of ginkgo leaves extract (ginkgo flavonoid 24% and ginkgolide 6%) and 1,000 parts of Korean red ginseng extract.

The preparation methods of ginkgo leaves extract and Korean red ginseng extract were the same as in Example 1.

### EXAMPLE 4

A composition for treating attention deficit hyperactivity disorder in children, including the following components in parts by mass:
25 parts of phosphatidylserine (20%), 1 parts of ginkgo leaves extract (ginkgo flavonoid 24% and ginkgolide 6%) and 500 parts of Korean red ginseng extract.

The preparation methods of ginkgo leaves extract and Korean red ginseng extract were the same as in Example 1.

### EXAMPLE 5

A soft capsule and its preparation method is described in this example, and the soft capsule was composed of the composition in Example 1 and excipients.

1. The parts by mass was converted into specific mass, and the formula of soft capsules was as follows:

The core active ingredients of each soft capsule were composed of: phosphatidylserine (20%) 50 mg, ginkgo leaves extract (ginkgo flavonoid 24% and ginkgolide 6%) 62.5 mg and Korean ginseng extract 250 mg.

The excipients of each soft capsule were composed of: flaxseed oil 180 mg, beeswax 20.00 mg, gelatin 210.00 mg, glycerol 100.00 mg, water 38.00 mg, titanium dioxide 3.50 mg and cocoa shell pigment 0.42 mg.

2. The preparation steps were as follows:
1) ginkgo leaves extract and Korean red ginseng extract were grinded into fine powder and passed through a 60-mesh sieve to obtain ginkgo leaves extract powder and Korean red ginseng extract powder respectively;
2) phosphatidylserine, ginkgo leaves extract powder and Korean red ginseng extract powder were mixed uniformly and passed through a 60-mesh sieve, the undersize materials were collected to obtain mixed components;
3) the mixed components in step 2) were uniformly mixed with flaxseed oil and beeswax to obtain an oily mixture;
4) gelatin, glycerol, water, titanium dioxide and cocoa shell pigment were evenly mixed to prepare a glue solution, and the oily mixture prepared in step 3) was thermal encapsulated while it is hot, and the resulting encapsulated glue solution was then cooled and demoulded to obtain a soft capsule. The specific steps to prepare soft capsule were as follows:

The glue solution and the oily mixture were spray out through the nozzle of the pill dropping machine, the oily mixture in the inner layer was encapsulated with the outer glue solution according to the routine operation procedure of the pill dropping machine, and the resulting encapsulated glue solution was then dropped into the liquid paraffin coolant, where the cooled glue solution formed a sphere due to the surface tension and gradually solidified into soft capsules.
5) the soft capsules obtained in step 4) were packed with 200 cc PE bottles to obtain finished soft capsule products.

### EXAMPLE 6

A soft capsule and its preparation method is described in this example, and the soft capsule was composed of the composition in Example 2 and excipients.

1. The parts by mass was converted into specific mass, and the formula of soft capsules was as follows:

The core active ingredients of each soft capsule were composed of: phosphatidylserine (20%) 10 mg, ginkgo leaves extract (ginkgo flavonoid 24% and ginkgolide 6%) 200 mg and Korean ginseng extract 1 mg.

The excipients of each soft capsule were composed of: flaxseed oil 250 mg, beeswax 10.00 mg, gelatin 100.00 mg, glycerol 50.00 mg, water 19.00 mg, titanium dioxide 2.00 mg and cocoa shell pigment 0.20 mg.

2. The preparation method was the same as in Example 5.

### EXAMPLE 7

A soft capsule and its preparation method is described in this example, and the soft capsule was composed of the composition in Example 3 and excipients.

1. The parts by mass was converted into specific mass, and the formula of soft capsules was as follows:

The core active ingredients of each soft capsule were composed of: phosphatidylserine (20%) 1 mg, ginkgo leaves extract (ginkgo flavonoid 24% and ginkgolide 6%) 100 mg and Korean ginseng extract 1,000 mg.

The excipients of each soft capsule were composed of: flaxseed oil 100 mg, beeswax 5 mg, gelatin 50 mg, glycerol 100 mg, water 30.00 mg, titanium dioxide 3.00 mg and cocoa shell pigment 0.20 mg.

2. The preparation method is the same as in Example 5.

### EXAMPLE 8

A soft capsule and its preparation method, the soft capsule was composed of the composition in Example 4 and excipients.

1. The parts by mass was converted into specific mass, and the formula of soft capsules was as follows:

The core active ingredients of each soft capsule were composed of: phosphatidylserine (20%) 25 mg, ginkgo leaves extract (ginkgo flavonoid 24% and ginkgolide 6%) 1 mg and Korean ginseng extract 500 mg.

The excipients of each soft capsule were composed of: flaxseed oil 10 mg, beeswax 10.00 mg, gelatin 50.00 mg, glycerol 100.00 mg, water 38.00 mg, titanium dioxide 3.50 mg and cocoa shell pigment 0.30 mg.

2. The preparation method was the same as in Example 5.

### Application Example 1

Tests on improving attention deficit hyperactivity disorder in children were carried out with the composition in Example 1, and the specific steps were as follows:

Spontaneously hypertensive rats (SHR) were randomly divided into two groups according to their body weights, with 8 rats in each group, namely the experimental group and the control group, and the body weight between rats did not exceed 10 g to avoid the error caused by large gap.

The experimental group was gavaged with 1 mL of 1 mg/mL sterile aqueous solution of the mixture composed of phosphatidylserine, ginkgo leaves extract and Korean red ginseng extract according to the proportion in Example 1, while the experimental group was gavaged with 1 mL of sterile water. 21 consecutive days.

The degree of spontaneous activity, social preference, irritability, attention transfer and spatial memory of the SHR were observed.

(1) The degree of autonomic activity: the autonomic activity of rats was tested at 8:00 pm on the 16th day of drug administration. During the test, rats were placed into an Open Field Test box (44 cm×44 cm×54 cm), first adapted to the environment for 1 min, and then observed for 30 min. The observed video files were analyzed by EthoVision XT 9.0, an animal behavior tracker of Noldus. Behavioral indexes of each rat, total moving distance, moving speed, stay time in the central area, central area distance and activity continuity, were recorded. The results are shown in Table 1:

TABLE 1
Effect of Formula on Autonomic Activity of ADHD Model Rats

| | | Moving | Stay time in | | |
|---|---|---|---|---|---|
| | Total moving | speed/ | the central | Central area | Activity |
| Group | distance/cm | (cm/s) | area/s | distance/cm | continuity |
| Experimental group | 5409.77 ± 837.45 | 3.89 ± 0.47 | 90.40 ± 76.17 | 1893.8 ± 717.51 | 10.96 ± 2.02 |
| Control group | 6701 ± 567.24 | 7.21 ± 0.50 | 103.86 ± 78.97 | 2025.33 ± 696.38 | 15.06 ± 1.97 |

From Table 1, it can be concluded that compared with the control group the total moving distance, moving speed and activity continuity of rats in the experimental group were significantly reduced.

(2) Degree of social preference: at 20:00 on the 17th day of drug administration, rats were placed into the boxes of three-chamber social test for social novelty preference test, strange rats were placed into one side of the device in advance, and the other side was not placed on the rat as the blank side. During the test, the animals were first adapted to the environment for 1 min, and then observed for 30 min. Detection indexes: the time of the strange rat side of the animal to be tested (the time and time proportion of the rats to be tested on the strange rat side) and the time percentage of the strange rat side (the time of the rats to be tested on the strange rat side/the time of independent activities×100%). The results are shown in Table 2:

TABLE 2
Effect of Formula on Social Ability of ADHD Model Rats

| | Time of the strange | Time percentage of the |
|---|---|---|
| Group | rat side/s | strange rat side/% |
| Experimental group | 896.35 ± 368.24 | 225.69 ± 36.94 |
| Control group | 736.31 ± 256.84 | 110.26 ± 56.34 |

From Table 2, it can be concluded that compared with the control group the components contained in the formula can significantly improve the social ability of rats.

(3) Irritability: irritability test was conducted on the 20th day after administration, and the irritability-like behaviors were detected by measuring the aggressive response. The specific operation and statistical methods were detailed in the references (Adam K, Giordano DG, Jenni K, et al. CRF1 receptor-dependent increases in irritability-like behavior during abstinence from chronic intermittent ethanol vapor exposure [J]. Alcohol Clin Exp Res, 2017, 4 (11): 1886-1895), the results are shown in Table 3:

TABLE 3
Effect of Formula on Aggressive Behavior of ADHD Model Rats

| | Total aggressive behaviors/ |
|---|---|
| Group | number of times |
| Experimental group | 4.72 ± 2.63 |
| Control group | 8.56 ± 1.29 |

It can be concluded from Table 3 that compared with the control group, the formula in the experimental group could significantly reduce the number of aggressive behaviors of rats and improve their irritability and other characteristics.

(4) Spatial memory ability: Barnes maze test was divided into learning process (16th-18th day) and test process (19th day), during the test, light stimulation was used as the motivation to drive rats to enter the target hole. The specific training and testing methods were detailed in the references (Duan Shangchun, Qing Wenxiang, Wang Xueqin, et at., Inhibiting RIP1 improves cognitive impairments induced by chronic stress in mice [J]. Chinese Journal of Anatomy, 2018, 41(5): 547-552), the results are shown in Table 4.

TABLE 4
Effect of formula on spatial memory ability of ADHD model rats

| Group | incubation period/s | mistakes/number of times |
|---|---|---|
| Experimental group | 13.98 ± 4.79 | 1.72 ± 1.23 |
| Control group | 15.29 ± 5.95 | 2.32 ± 1.34 |

From Table 4, it can be concluded that compared with the control group, the rats in the experimental group could significantly reduce the number of mistakes, which suggested that the formula had significant ability to improve spatial memory.

It can be concluded from the above examples that the composition has a good therapeutic effect on attention deficit hyperactivity disorder in children, has a good improvement effect on various mal-adaptations of attention deficit hyperactivity disorder in children, and can reduce the use of stimulants. Through test observation, it is found that the activities, eating and defecation of rats fall within the normal ranges, and no side effects are found, therefore, the composition of the present disclosure can reduce the incidence of various side effects.

Although the above embodiments have described the present disclosure in detail, they are only a part of the embodiments of the present disclosure, not the whole embodiments. Other embodiments can also be obtained by those skilled in the art without inventive step based on the present embodiments, all of which should also fall within the scope of protection of the present disclosure.

What is claimed is:

1. A soft capsule, wherein the soft capsule comprises a composition and a soft capsule excipient, wherein the composition comprises the following components in parts by mass: 1-50 parts of phosphatidylserine, 1-200 parts of ginkgo leaves extract, and 1-1,000 parts of red ginseng extract;
wherein a mass ratio of phosphatidylserine to soft capsule excipient is (1-50): (0-650), and the mass of soft capsule excipient is not 0; and
wherein the soft capsule excipient comprises flaxseed oil, beeswax, gelatin, glycerol, water, titanium dioxide and pigment.

2. The soft capsule according to claim 1, wherein the soft capsule excipient comprises 0-250 parts of flaxseed oil, 0-20 parts of beeswax, 0-210 parts of gelatin, 0-100 parts of glycerol, 0-38 parts of water, 0-3.50 parts of titanium dioxide and 0.42 parts of pigment in parts by mass; wherein the pigment comprises cocoa shell pigment; and wherein the mass of flaxseed oil, beeswax, gelatin, glycerol, water, titanium dioxide and pigment is not 0.

3. A method for preparing the soft capsule according to claim 1, wherein the method comprises the following steps:
mixing phosphatidylserine, ginkgo leaves extract powder and red ginseng extract powder to obtain a mixture;
mixing the mixture, flaxseed oil and beeswax to obtain an oily mixture;
mixing gelatin, glycerol, water, titanium dioxide and pigment to obtain a glue solution; and
thermally encapsulating the oily mixture with the glue solution and cooling to obtain a soft capsule.

4. A method for treating or adjuvant treating attention deficit hyperactivity disorder in children, comprising administering the soft capsule according to claim 1 to a subject in need.

5. The method according to claim 4, wherein the treating or adjuvant treating comprise one or more of maintaining nervous system, anti-inflammatory, improving body energy metabolism, repairing neurotransmitter conduction, improving learning and memory ability, and alleviating the mal-adaptations of attention deficit hyperactivity disorder in children; and
wherein the alleviating the mal-adaptation symptoms of attention deficit hyperactivity disorder in children comprises one or more of improving attention, adjusting emotion, improving hyperactivity symptoms, improving impulsive symptoms and reducing aggression.

6. The soft capsule according to claim 1, wherein the soft capsule is administered in combination with stimulants as a combined drug for treating attention deficit hyperactivity disorder in children.

7. The soft capsule according to claim 1, wherein a purity of phosphatidylserine is at least 20%.

8. The soft capsule according to claim 1, wherein the ginkgo leaves extract comprises ginkgo flavonoid and ginkgolide, and a mass concentration of ginkgo flavonoid is 24%, and a mass concentration of ginkgolide is 6%.

9. The soft capsule according to claim 1, wherein the red ginseng extract comprises Korean red ginseng extract.

10. The method according to claim 3, wherein the soft capsule excipient comprises 0-250 parts of flaxseed oil, 0-20 parts of beeswax, 0-210 parts of gelatin, 0-100 parts of glycerol, 0-38 parts of water, 0-3.50 parts of titanium dioxide and 0.42 parts of pigment in parts by mass; wherein the pigment comprises cocoa shell pigment; and wherein the mass of flaxseed oil, beeswax, gelatin, glycerol, water, titanium dioxide and pigment is not 0.
